(19) United States
(12) Patent Application Publication
(10) Pub. No.: US 2011/0083495 A1
(43) Pub. Date: Apr. 14, 2011

(54) DEVICES AND METHODS FOR COLLECTING AND PROCESSING A SPECIMEN

(76) Inventors: Val L. Poll, Ogden, UT (US); Steven J. MIller, Hyrum, UT (US); Jay J. Davis, Smithfield, UT (US); Brian T. Leishman, Wellsville, UT (US); Joshua L. Kirk, Providence, UT (US)

(21) Appl. No.: 12/766,786

(22) Filed: Apr. 23, 2010

Related U.S. Application Data
(63) Continuation-in-part of application No. 12/577,560, filed on Oct. 12, 2009.

(51) Int. Cl. G01N 1/28; G01N 1/02; G01N 33/00

(52) U.S. Cl. 73/64.56; 73/863.23

(57) ABSTRACT

Embodiments of the present invention include a specimen collection assembly that limits or eliminates the need for transferring a specimen sample and/or solution mixed with the specimen sample from one container to another. The specimen collection assembly includes a specimen collection member, a filter and a lid each to be employed with a vile. The filter is attachable to a distal portion of the specimen collection member. The lid is configured to be positioned over the open end of the vile with both the specimen collection member and filter positioned in the vile. With this arrangement, a liquid solution can be introduced into the vile and then agitated to mix with a specimen sample. The fecal loop with the attached filter can then be withdrawn from the vile to leave the remaining filtered and mixed liquid solution in the vile.

### CROSS-REFERENCE TO RELATED APPLICATION

This application is a continuation-in-part of U.S. patent application Ser. No. 12/577,560, filed Oct. 12, 2009, entitled “DEVICES AND METHODS FOR COLLECTING AND PROCESSING A SPECIMEN,” the disclosure of which is incorporated by reference herein in its entirety.

### TECHNICAL FIELD

The present invention relates generally to a device, assembly and method employed in the collection and analysis of specimens and, more specifically, the collection and analysis of fecal matter or other biological matter.

### BACKGROUND

Various methods and devices have been used in the past for collecting, transporting and then testing biomedical samples. Many of the biomedical samples are fecal samples taken by veterinarians or technicians at veterinary clinics. The standard practice for taking a fecal sample involves transferring the sample from one container to another typically several times. For example, after extracting a sample from a mammalian, such as with a fecal loop, the veterinarian will then empty the sample from the fecal loop into a container or place the fecal loop with the sample in the container. Such container is then transported to a laboratory for testing. Once the laboratory receives the shipment, the sample is then placed into a mixing container, in which the sample is mixed with a liquid solution. The liquid solution is then strained or filtered into another container or vile for conducting testing on the filtered solution. As such, from the field to obtaining results from laboratory testing, the sample is typically transferred into different containers several times. Such transferring of the sample is tedious, messy, inefficient and costly, especially when compounding this process with high sample volumes being received at laboratories conducting the sample testing. Further, each time the sample is transferred from one container to another there is an increased risk of compromising the sample and/or contamination from outside sources. Unfortunately, the high volume of samples being tested in laboratories even further compounds the risk of compromising the sample specimens.

Based on the foregoing reasons, it would be advantageous to provide a sample collection assembly that limits or, even eliminates, the risk of compromising or contamination of a specimen sample due to transferring such specimen sample from one container to another. Further, it would be advantageous to provide a sample collection assembly that is less tedious and messy and improves efficiency for the laboratory technician and, therefore, saves time and is less costly.

### BRIEF SUMMARY OF THE INVENTION

Embodiments of the present invention include a specimen collection assembly that limits or eliminates the need for transferring a specimen sample and/or solution mixed with the specimen sample from one container to another from the collection process to the testing of the specimen in the laboratory. Embodiments of the present invention further include related methods and systems.

In accordance with one embodiment of the present invention, a specimen collection assembly configured to be used with a container member having an open end and a closed end is provided. The specimen collection assembly includes a specimen collection member, a filter and a lid. The specimen collection member includes a collection portion and a handle, in which the collection portion extends from one end of the handle. The filter is sized and configured to be positioned in the container member and is configured to be attached to the collection portion in the container member, in which the filter is configured to surround at least a distal portion of the collection portion. The lid is configured to be positioned over the open end of the container member with the filter and the specimen collection member positioned in the container member.

In one embodiment, the filter includes a central portion and a filter portion, the central portion being configured to interconnect with the collection portion and the filter portion configured to surround at least the distal portion of the collection portion, the filter portion including multiple openings extending therethrough. In anther embodiment, the filter includes a central portion and multiple filter tabs extending from the central portion, the multiple filter tabs configured to be deflectable between a first configuration and a second configuration, in which the multiple filter tabs are deflectable to the second configuration upon being positioned in the container member.

In still another embodiment, the filter includes an extension nub extending therefrom that is configured to attach to a distal end of the collection portion. Such collection portion includes a hole defined therein sized and configured to receive the extension nub. With this arrangement, the collection portion is reversibly attachable to the filter with a snap-type fit.

In accordance with another embodiment of the present invention, a method for processing a specimen with a vile having an open end and a closed end is provided. The method includes: providing a specimen collection assembly, the assembly including a specimen collection member, a filter and a lid, the specimen collection member having a collection portion and a handle, the lid configured to cover the open end of the vile; capturing a specimen with the collection portion of the specimen collection member; connecting a distal end of the collection portion to the filter; and inserting the filter with the connected specimen collection member into the vile so that at least a portion of the filter is positioned distal the collection portion within the vile.

In one embodiment, the method also includes covering the vile with the lid so that a proximal end of the handle extends into a hollow portion defined in the lid. In another embodiment, the method also includes transporting the specimen collection assembly to another location to analyze the specimen. Further, the method also may include inserting a solution through an opening in the lid to at least partially fill the vile with the solution while the lid remains captured over the vile. In addition, the method may also include inserting a solution through an opening in the lid so that the solution enters the vile by flowing through channels defined in a proximal end of the handle of the specimen collection member. Further, the method may also include mixing the solution with the specimen with each of the specimen collection member and the filter captured in the vile and the lid covering the open end of the vile. The method further includes removing the specimen collection member and filter from the vile while maintaining interconnection between the specimen collection member and the filter. The method further includes the removing step including maintaining the mixed solution in the vile.

In another embodiment, the inserting step includes deflecting multiple filter portions upward while inserting the filter through the open end of the vile. Also, in another embodiment, the inserting step includes deflecting the multiple filter portions upward while inserting the filter through the open end of the vile. The deflecting step may include surrounding at least a distal portion of the collection portion with the central portion adjacent the distal end of the collection portion and the multiple filter portions deflected and positioned around at least the distal portion of the collection portion.

In still another embodiment, the connecting method step includes manually attaching the filter to the distal end of the collection portion with a reversible attachment. The connecting step may also include inserting an extension portion extending from the filter into a hole defined in the distal end of the collection portion.

In accordance with another embodiment of the present invention, a method for processing a specimen with a single vile having an open end and a closed end is provided. The method includes: providing a specimen captured in a vile with a specimen collection assembly, the specimen collection assembly including a specimen collection member, a filter and a lid, the specimen collection member including a collection portion with the specimen captured therewith the filter removably attached to a distal end of the collection portion and positioned to surround at least a distal portion of the collection portion, and the lid covering an open end of the vile; inserting a solution through an opening in the lid to at least partially fill the vile; mixing the solution with the specimen with each of the specimen collection member and the filter captured in the vile and the lid covering the open end of the vile; removing the specimen collection member and filter from the vile while maintaining interconnection between the specimen collection member and the filter; and testing the mixed solution from the vile.

In one embodiment, the providing method step includes providing the lid having a hollow portion defined therein for receiving a proximal end of a handle of the specimen collection member. In another embodiment, the inserting method step includes inserting the solution through the opening and hollow portion defined in the lid so that the solution enters the vile by flowing through channels defined in the proximal end of the handle of the specimen collection member.

These and other aspects of the present invention will become more fully apparent from the following description and appended claims, or may be learned by the practice of the invention as set forth hereinafter.

### BRIEF DESCRIPTION OF THE SEVERAL VIEWS OF THE DRAWINGS

The foregoing and other advantages of the invention will become apparent upon reading the following detailed description and upon reference to the drawings in which:

FIG. 1 is an exploded view of a fecal specimen collection assembly, illustrating a fecal loop and a filter configured to be placed within a vile and a lid to cover the vile, according to an embodiment of the present invention;

FIG. 2 is a perspective view of the fecal specimen collection assembly of FIG. 1 in, an assembled state, according to the present invention;

FIG. 3A is a cross-sectional view of a distal portion of the fecal specimen collection assembly depicted in FIG. 2, illustrating the fecal loop attached to the filter at a bottom portion of the vile, according to one embodiment of the present invention;

FIG. 3B is a cross-sectional view of a proximal portion of the fecal specimen collection assembly depicted in FIG. 2, illustrating the lid captured over the vile and handle of the fecal loop, according to another embodiment of the present invention;

FIG. 4 is a front view of the filter in a non-deflected position, according to another embodiment of the present invention;

FIG. 4A is a cross-sectional view of the filter taken along line 4A of FIG. 4, depicting the filter being moveable to a deflected position, according to another embodiment of the present invention;

FIG. 5 is a perspective view of the filter attached to a distal end of the fecal loop, the filter in a non-deflected position, according to another embodiment of the present invention;

FIG. 5A is a front view of the filter attached to a distal end of the fecal loop, depicting the filter within the vile (shown in outline), according to another embodiment of the present invention;

FIG. 6 is a perspective view of a proximal portion of the handle of the fecal loop, depicting channels formed in the proximal portion, according to another embodiment of the present invention;

FIG. 7 is a perspective view of a portion of the proximal portion of the handle captured within the lid, depicting a portion of a channel defined in the handle exposed with respect to the lid, according to another embodiment of the present invention;

FIG. 8A is a cross-sectional view of a distal portion of an assembled specimen collection assembly, illustrating a specimen collection member attached to a filter at a bottom portion of a vile, according to another embodiment of the present invention;

FIG. 8B is a cross-sectional view of a proximal portion of an assembled specimen collection assembly, illustrating a lid captured over a vile and handle of a specimen collection member, according to another embodiment of the present invention;

FIG. 9 is a perspective view of a specimen collection member and a filter in an unattached state, according to an embodiment of the present invention; and

FIG. 10 is a perspective view of the lid, according to an embodiment of the present invention.

### DETAILED DESCRIPTION OF THE INVENTION

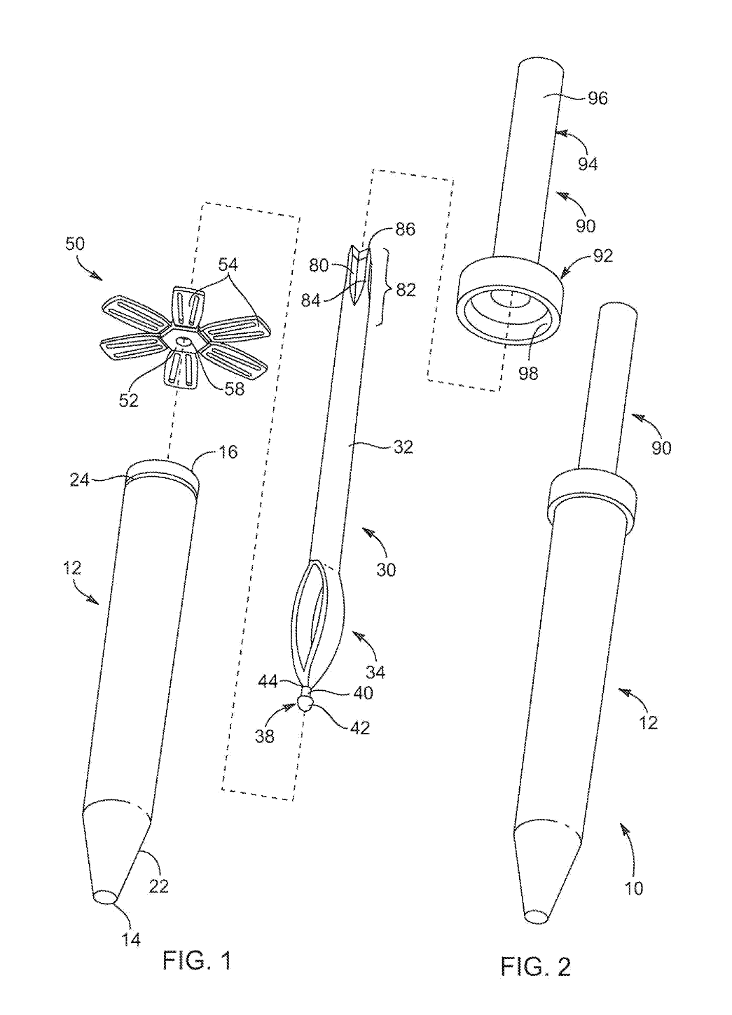

Referring to FIGS. 1 and 2, a specimen collection assembly 10 is shown, in exploded form and assembled, respectively. The specimen collection assembly 10 of the present invention is employed to minimize the steps and cost in the collection and processing of specimen samples, such as fecal matter specimens, in the field and laboratory. The specimen collection assembly 10 also minimizes the risk of compromising and/or contamination of a specimen sample due to transferring such specimen sample from one container to another.

Such a specimen collection assembly 10 can include a specimen collection member 30, a filter 50, and a lid 90, each to be employed with a typical vile 12 for containing the assembly and specimen. In particular, the specimen collection member 30 is employed first to obtain a specimen, such as a fecal specimen, or any other suitable specimen for sampling. The specimen collection member 30 may then be attached to the filter 50. The specimen collection member 30 and attached filter 50 may then be inserted into the vile 12, after which, the lid 90 may then be placed over the vile 12 for retaining the assembled filter 50 and collection member 30, and specimen therein. The specimen with the specimen collection assembly 10 can then be transported to the laboratory for processing and testing. Once received, a laboratory technician can insert a solution into the vile 12 without removing the lid 90. The solution is then agitated or mixed with the specimen, after which, the specimen collection assembly 10 is removed from the vile 12. When removing the lid 90, the specimen collection member 30 and filter 50 are also removed such that the filter removes the larger matter from the specimen, leaving the filtered solution in the vile 12 for the laboratory technician to conduct further tests. With this arrangement, the specimen collection assembly of the present invention provides a method by which a single container may be used in the field and laboratory to perform laboratory tests on a specimen sample to, thereby, limit the risk of contamination of the sample and obtaining compromised or inaccurate test results. Further embodiments and structural details of the specimen collection assembly of the present invention will now be discussed.

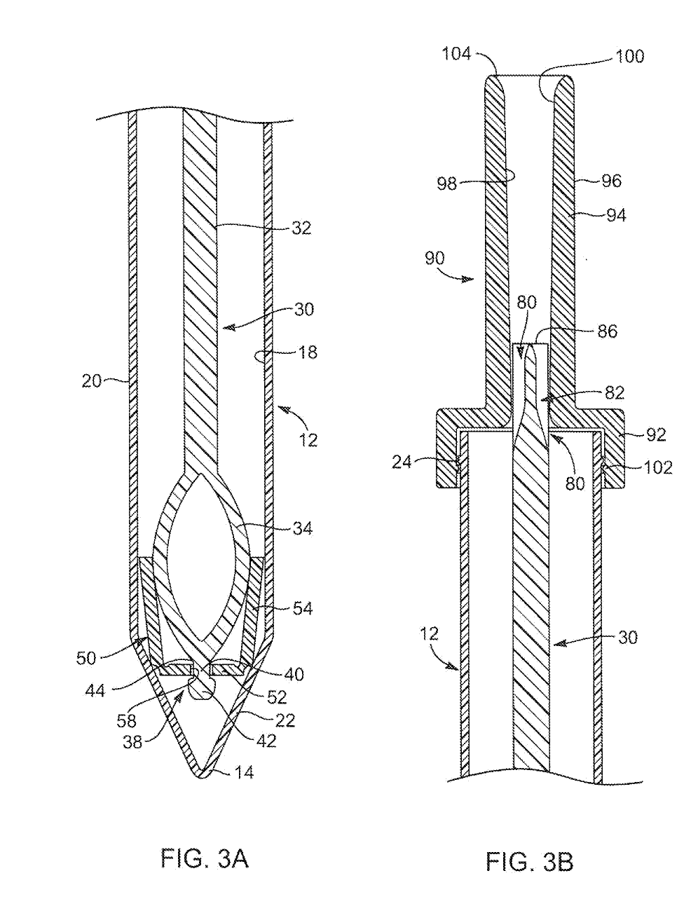

With reference to FIGS. 1 and 3A, the vile 12 or container member can typically include a hollow cylindrical shape or tubular member with a closed end 14 and an open end 16. The vile 12 includes an inner surface 18 and an outer surface 20 that may include a taper 22 adjacent the closed end 14 in a conical type shape. The outer surface 20 may include a protrusion 24 in the form of a ring extending adjacently around the open end 16. Such a protrusion 24 can assist in retaining the lid 90 to the proximal open end 16 of the vile 12.

The specimen collection member 30 can include a handle 32 and a specimen collection portion or, for example, a loop portion 34 that extends from one end of the handle 32. The specimen collection member 30 is configured to collect various types of specimens that may include fecal matter, or any other type of matter needed for testing. The specimen collection member 30 may include the basic features of a typical fecal loop, however, the specimen collection member 30 is not limited to a fecal loop. Such specimen collection member 30 may include other structures, such as a swab, or any other structure suitable for taking a specimen sample.

As set forth, the specimen collection portion can be in the form, and include the profile, of an oval loop or loop portion 34 extending from one end of the handle 32. Such a handle 32 and loop portion 34 may be sized and configured to readily allow a person to grasp the handle 32 and take, for example, a fecal sample from a mammalian body, with the loop portion 34. Although the specimen collection portion is shown as a loop structure, other suitable structures may be employed that readily capture fecal matter (or any other type of specimen) and substantially retain such fecal matter therewith. According to the present invention, in addition to the loop portion 34 and the handle 32, the specimen collection member 30 can include additional structural features designed to stream-line the collection and processing of a specimen.

With respect to FIGS. 1, 3A and 5, according to one embodiment, the specimen collection member 30 can include an attachment portion that is sized and configured to attach to the filter 50 such that the filter 50 is reversibly attached and at least a portion is positioned distal or surrounds a distal portion of the loop portion 34 of the specimen collection member 30. In one embodiment, the attachment portion of the specimen collection member 30 can be an extension nub 38. Such an extension nub 38 can include a neck 40 and a head 43 that can extend distally from a distal end 44 of the loop portion 34. The extension nub 38 can be sized and configured to be attached to the filter 50 with a snap-type fit or an interference type fit. In another embodiment, the extension nub 38 may include threads to be threadably attached to the filter or, any other suitable structure for attaching the specimen collection member 30 to the filter 50.

Now referring to FIGS. 1, 3A, 4 and 5, the filter 50 can include a central portion 52 and multiple filter tabs 54 extending from the central portion 52. As previously set forth, the filter 50 can include a filter attachment portion. In one embodiment, the filter attachment portion can include an aperture 58 or through hole defined in the central portion 52 of the filter 50 that is sized and configured to receive the extension nub 38 therein. The head 42 of the extension nub 38 can be manually forced through the aperture 58 with a snap-type fit or interference fit so that the distal end 44 of the loop portion 34 seats against a surface of the central portion 52 with the neck 40 positioned within the aperture 58 and the head 42 positioned therethrough.

In another embodiment, the filter attachment portion may be a recess sized and configured to receive the extension nub, or any other suitable structure for receiving the extension nub to attach the filter 50 to the specimen collection member 30. In another embodiment, the filter attachment portion may include an extension structure, or any other suitable structure, that is sized and configured to attach to a recess or capturing portion defined in the specimen collection member 30.

With respect to FIGS. 3A, 4, 4A and 5, as previously indicated, the filter 50 can include multiple filter tabs 54 or multiple filter portions that extend from the central portion 52. Further, the filter 50 includes a first side 60 and a second side 62 that define a periphery 64 or edge between the first side 60 and second side 62. The structure of the first side 60 and second side 62 may be formed to be substantially similar. As earlier indicated, the central portion 52 may include the aperture 58 or filter attachment portion, which may be positioned centrally within such central portion 52. The filter tabs 54 can extend radially from the central portion 52 to, collectively provide, a spoke-like configuration. For example, the central portion 52 can include a hexagonal configuration or six sides each with a filter tab 54 extending therefrom. The central portion 52 may include any other suitable shaped configuration, such as octagonal, square or round. Each filter tab 54 can include one or more openings 66 defined therein that extend through the tab 54 and may be sized and configured to facilitate a filtering function of the specimen. The openings 66 in the tab 54 can define an elongated shape with a width 68, or any other shape or configuration, to provide desired filtering characteristics.

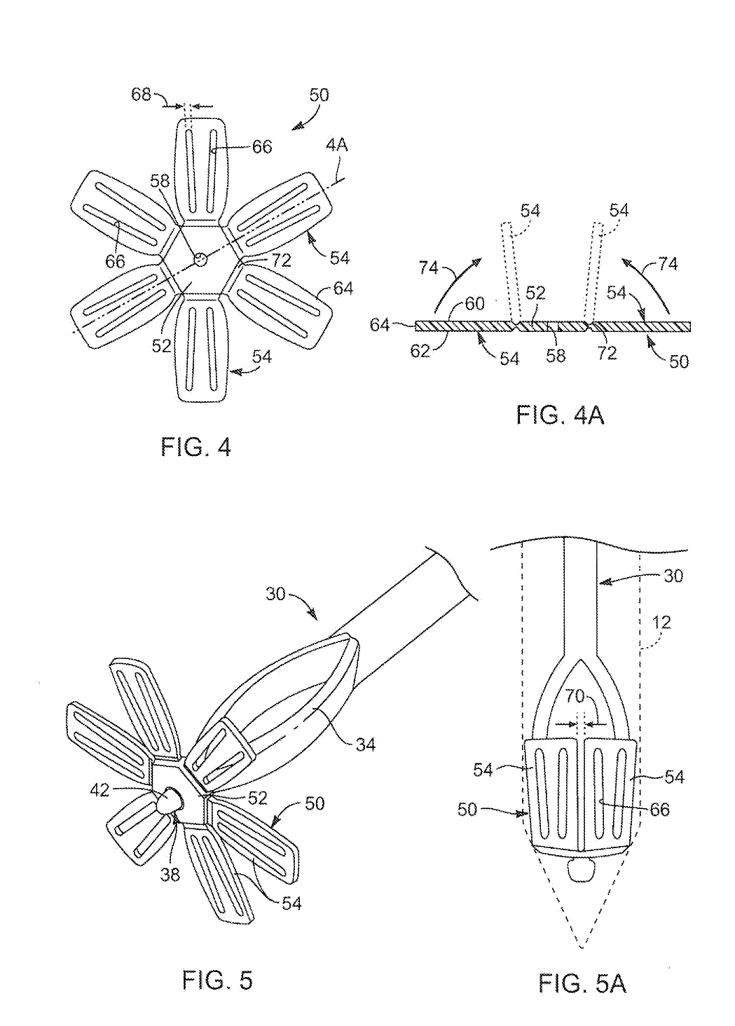

Further, with respect to FIGS. 4 and 5A, the filter tabs 54, when in a deflected configuration positioned in the vile 12 (shown in outline), may be spaced a distance 70 similar to the width 68 of the openings 66, such that the spaced distance 70 of the filter tabs 54 also provide a filtering function, similar to the openings 66 defined in the filter tabs 54. As shown in FIG. 4, the side walls or periphery 64 of each filter tab 54 may be slightly curved so that when placed in the vile 12 the spaced distance 70 is substantially consistent along a length of each filter tab 54, as shown in FIG. 5A. The width 68 of the openings 66 and the spaced distance 70 of two adjacent tabs may be about 0.0200 inches to 0.0400 inches and preferably about 0.0250 inches to 0.0350 inches. In this manner, both the openings 66 in the tabs 54 and the spaced distance 70 between the tabs 54 in the deflected configuration may provide the filtering function of the filter 50. In another embodiment, the filter tabs 54 or filter portion may also include a screen member or portions of a screen configuration.

As previously set forth, the multiple filter tabs 54 can be positioned in an expanded configuration or orientation or non-use state (depicted in FIGS. 4 and 5), and can also be positioned in a deflected configuration or orientation or a narrow confined, use state (depicted in FIGS. 3A and 5A). The filter 50 can be employed to readily deflect by forming the interconnection between the tabs 54 and the central portion 52 with a thinned wall structure 72. Deflection of the tabs 54 from the expanded configuration, deflecting relative to the central portion 52 or thinned wall structure 72, is shown with arrows 74 in FIG. 4A. With this arrangement, the thinned wall structure 72 provides a living hinge between the tabs 54 and the central portion 52 that may readily allow the tabs 54 to deflect to a deflected or narrow configuration when placing or, rather, inserting the filter 50 into the vile 12 while attached to the specimen collection member 30. Further, due to the first side 60 and second side 62 of the filter 50 being substantially similar, the filter 50 can be attached to the specimen collection member 30 at either the first side 60 or the second side 62 of the filter 50.

In another embodiment, the filter 50 can be preformed into the narrow configuration or use state (similar to that shown in FIG. 5A) such that the filter tabs 54 or filter portion is interconnected and do not deflect in the manner previously set forth. With this arrangement, the filter 50 may include a cup-like configuration with the central portion at a bottom wall of the cup-like configuration and side walls being the filter portion. As such, the extension nub can reversibly attach, for example, to the bottom wall of the filter such that the sidewall or filter portion surrounds at least a distal portion of the loop portion of the specimen collection member, as depicted in FIG. 5A.

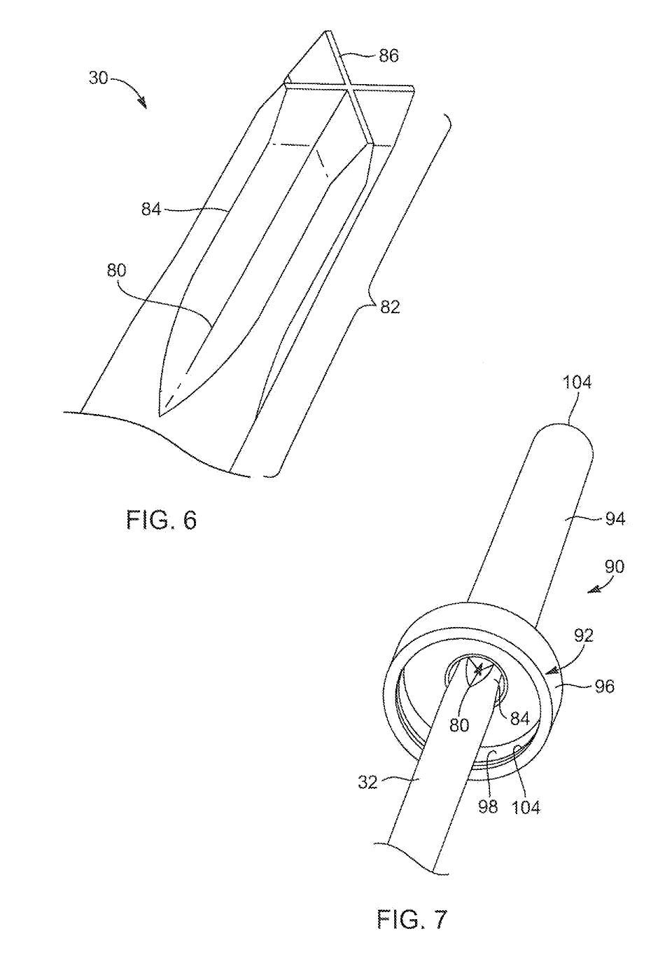

Referring now to FIGS. 1, 3B and 6, in one embodiment, the specimen collection member 30 may also include one or more channels 80 defined in a proximal portion 82 of the handle 32 of the specimen collection member 30. Such one or more channels 80 are sized and configured to facilitate a liquid solution (not shown), employed during a testing procedure, to flow through the channels 80 when the specimen collection member 30 is captured in the vile 12 with the lid 90 positioned over the vile 12. The one or more channels 80 can extend and be defined in the handle 32 with a ravine-like structure or any other suitable structure, such as a tunnel, or any other type of passageway that will facilitate the liquid solution to pass therethrough. In one embodiment, the one or more channels 80 can include a plurality of channels, such as four channels. Each channel 80 can be defined by a ridge 84 extending along both sides of each channel 80. The channels 80 can extend along a longitudinal length of the handle 32 from a proximal end 86 and along the proximal portion 82 of the handle 32 or partially along the length of the handle 32.

Now referring to FIGS. 1, 3B and 7, in one embodiment, the lid 90 may include a lid head 92 and a lid extension 94. The lid head 92 is sized and configured to be captured over the open end 16 of the vile 12. The lid extension 94 is sized and configured to at least partially capture the proximal portion 82 of the handle 32 of the specimen collection member 30. The lid head 92 and the lid extension 94 can each include an outer surface 96 and an inner surface 98 such that the inner surface 98 defines a hollow portion 100 extending through the length of the lid extension 94 and extending between a proximal opening 104 and a distal opening106 to define a through hole in the lid 90. The distal opening 106 is defined in and exposed in the inner surface 98 of the lid head 92 and the proximal opening 104 is defined and exposed at a tip end 108 of the lid extension 94. The inner surface 98 of the lid head 92 can include a ridge 102 sized and configured to fit over the ring protrusion 24 of the vile 12 to assist maintaining the lid 90 with the vile 12. Further, the distal opening 106 and hollow portion 100 defined in the lid extension 94 may be sized and configured to fit, in a snug manner, with at least a portion of the proximal portion 82 of the handle 32 such that the ridges 84 at the proximal portion 82 engage or are in direct contact with the inner surface 98 of the lid extension 94.

As depicted in FIGS. 3B and 7, with the lid extension 94 sized and configured to at least partially receive the proximal portion 82 of the handle 32, a portion of the one or more channels 80 are exposed to, thereby, allow liquid solution to pass therethrough. With this arrangement, solution can be passed through the proximal opening 100 at the tip end 108 of the lid 90, in which the solution passes through the hollow portion 100 of the lid 90 and along the channels 80 and into the vile 12 without having to remove the lid 90.

Referring again to FIG. 1, the vile 12, specimen collection member 30, filter 50 and lid 90 can be formed from polymeric materials or thermo plastics, such as Polypropylene (PP), High Density Polyethylene (HDPE), Low Density Polyethylene (LDPE), or any other suitable material known to those of ordinary skill in the art. Further, such can be formed and manufactured utilizing manufacturing techniques known in the art, such as injection molding, thermo forming, plastic stamping, or machining, or any other suitable technique known in the art.

In use, the specimen collection assembly 10 of the present invention substantially reduces the risk of cross-contamination or compromising the specimen sample to be tested by limiting the need to transfer the specimen from one container to another in the field and while processing of the specimen sample. Specifically, in one embodiment, a technician, such as a veterinarian, takes a fecal sample, utilizing the specimen collection member 30. Once the technician obtains a suitable fecal sample, captured within the loop portion 34 of the specimen collection member 30, the technician then attaches the specimen collection member 30 to the filter 50. This attachment may be employed by manually inserting the extension nub 38 into the aperture 58 defined in the central portion 52 of the filter 50 until the extension nub 38 snaps into an attached position, as depicted in FIG. 5. At this stage, the filter 50 is in an expanded orientation. The technician may then insert the filter 50 and specimen collection member 30 into the vile, first inserting the bottom side of the filter 50, to thereby fold or deflect the filter tabs 54 into a deflected orientation, as depicted in FIGS. 3A and 5A. Once the filter 50 is positioned adjacent the closed end 14 within the vile 12 with the specimen collection member 30 attached thereto, a portion of the proximal portion 82 of the handle 32 of the specimen collection member 30 may be positioned to extend above the open end 16 of the vile 16, as depicted in FIGS. 3A and 3B. The lid 90 may then be captured or placed over the open end 16 of the vile. Such capturing may include at least a portion of the proximal portion 82 of the vile 12 being captured in the lid extension 94 of the lid 90 such that a portion of the channels 80 defined in the proximal portion 82 are exposed with respect to the lid extension 94, as depicted in FIGS. 3B and 7. At this stage, the specimen collection assembly 10 is assembled, as depicted in FIG. 2, and may then be transported to the laboratory for testing. Once a lab technician receives the specimen collection assembly 10, the lab technician may prepare to mix a liquid solution (not shown) with the fecal sample within the vile 12. Such can be employed without removing the lid 90 by inserting the liquid solution through the proximal opening 104 at the tip end 108 of the lid 90 (see FIG. 3B). The liquid solution enters the vile 12 through such opening 104 and along the channels 80 formed in the proximal portion 82 of the handle 32. Once the lab technician has filled the vile 12 with the required amount of liquid solution, the liquid solution is mixed with the fecal sample by shaking or agitating the vile 12. After agitation of the vile 12, the lid 90 is removed along with the specimen collection member 30 and attached filter 50. The position of the filter 50 is at least partially on a distal side or distal end 44 of the loop portion 34 bearing the fecal sample such that the filter 50 at least partially surrounds a distal portion of the loop portion 34, as depicted in FIGS. 3A and 5A. As such, when the filter 50 and specimen collection member 30 are removed, the filtered liquid solution (that has been mixed with the fecal matter) is left remaining in the vile 12. The filter 50 and attached specimen collection member 30 and lid 90 may then be properly discarded. The lab technician can then simply analyze the filtered solution left in the vile 12, the vile 12 being the original vile 12 utilized when taking the specimen sample. In this manner, the specimen collection assembly 10 of the present invention stream-lines the testing process of specimen samples by facilitating the use of a single vile 12 from collecting the specimen in the field through the processing and testing procedure to, thereby, limit risk associated with contamination and compromise of the specimen sample.

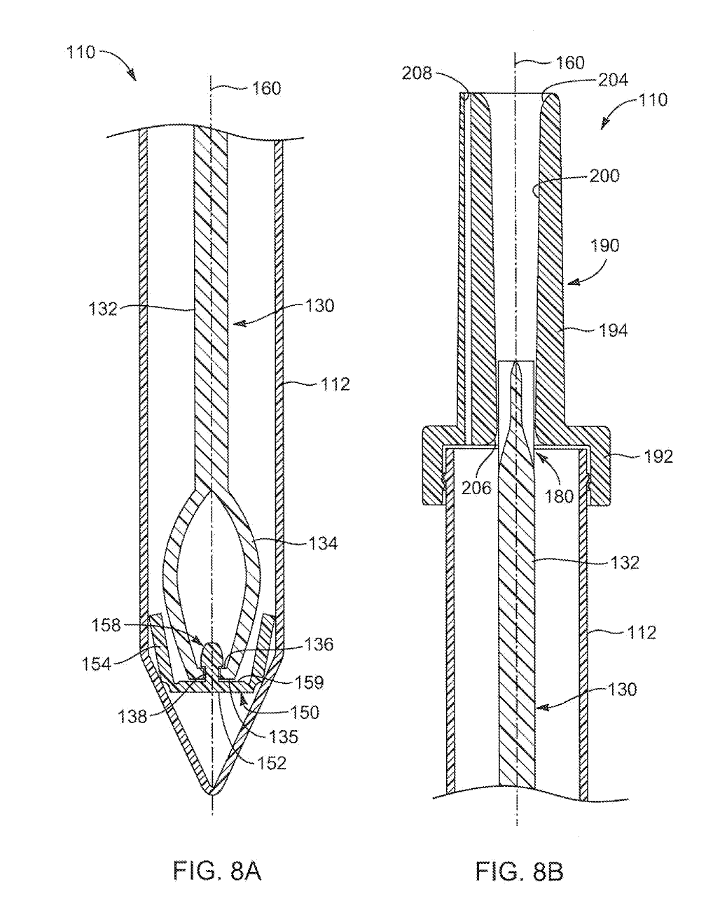

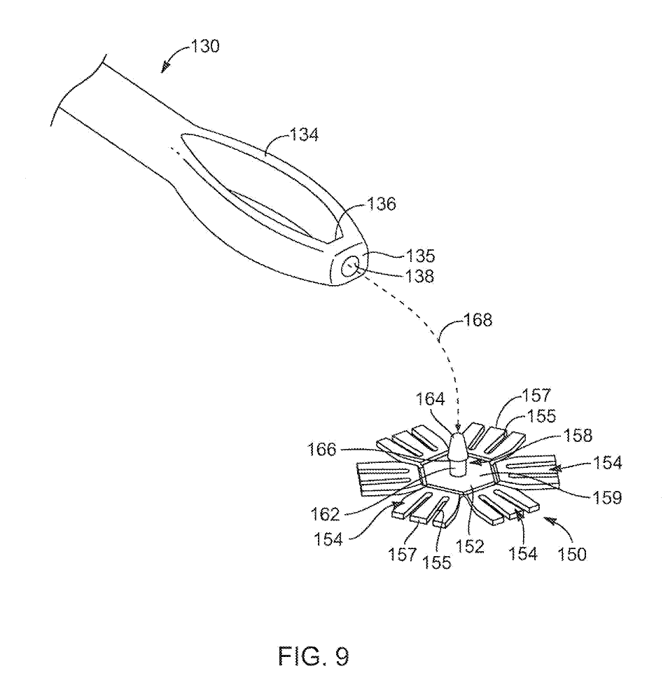

Referring now to FIGS. 8A and 8B, another embodiment of the specimen collection assembly 110 used with a vile 112, according to the present invention is shown. This embodiment may be similar to the embodiment depicted in FIG. 1, except for the interconnection between the specimen collection member 130 and the filter 150 and further, the lid 190 provides modified and/or additional structural features. In particular, as depicted in FIGS. 8A and 9, the loop portion 134, extending from one end of the handle 132 of the specimen collection member 130, may include a distal flat surface 135 (at the most distal end of the specimen collection member 130) that may define a hole 138 therein extending through a distal portion or distal end of the loop portion 134. In addition, the central portion 152 of the filter 150 may include an extension nub 158 extending from one side 159 of the filter 150. With this arrangement, the extension nub 158 of the filter 150 is sized and configured to facilitate attachment to the hole 138 defined in the loop portion 134 of the specimen collection member 130.

In one embodiment, the hole 138 defined in the loop portion 134 may extend between the flat distal surface 135 of the loop portion 134 to an inner surface 136 of the loop portion 134 to define the hole 138 extending therethrough. This hole 138 may extend axially or co-axially substantially along a longitudinal axis 160 of the specimen collection member 130 also depicted as the longitudinal axis 160 of the assembled specimen collection assembly 110. In another embodiment, the hole 138 may be an opening or recess defined in the distal flat surface 135 of the loop portion 134 sized and configured to receive and attach to the extension nub 158 of the filter 150. Such a recess may include ridges defined therein to assist in latching or grabbing to the extension nub 158 of the filter.

With reference to the filter 150, the extension nub 158 may include, similar to the extension nub of the previous embodiment, a neck 162 and a head 164 with a ridge 166 defined between the neck 162 and the head 164. The head 164 may include a somewhat oval or round shape with a head diameter dimension slightly larger than a diameter of the hole 138 defined in the loop portion 134 of the specimen collection member 130. Further, the neck 162 may include a neck height dimension slightly larger than a length dimension of the hole 138 with a neck diameter dimension slightly smaller than the diameter of the hole 138 defined in the loop portion 134. With this arrangement, the hole 138 of the specimen collection member 130 can be positioned over the extension nub 158 of the filter 150, as depicted by dotted line 168 in FIG. 9. The specimen collection member 130 may then be manually forced or pressed over the extension nub 158 of the filter 150 to, thereby, attach the specimen collection member 130 to the filter 150 with an interference type fit or snap-type fit. The head 164 of the extension nub 158, when sliding through the hole 138, may be configured to slightly deform, being made from a polymeric material, as such head 164 may be slightly larger than the hole 138, as previously indicated. As such, once the head 164 extends completely through the hole 138 so that the neck portion 162 is positioned within the hole 138, the ridge 166 between the head 164 and the neck 162 may be seated against the inner surface 136 of the loop portion 134. Further, attachment between the specimen collection member 130 and the filter 150 may be assessed once the distal flat surface 135 of the specimen collection member 130 abuts or is flush against the central portion 152 of the filter 150. In this manner, the interconnection between the filter 150 and the specimen collection member 130 may be an interference or snap-type fit that may readily facilitate manual disconnection if desired.

Further, as depicted in FIG. 9, another embodiment of the filter 150 is shown. In particular, the tabs 154 of the filter may define openings 155 therein that extend to an outer periphery 157 of the tabs 154. In one embodiment, the openings 155 may define an elongated channel like configuration. In other words, the openings 155 may extend to and may be exposed to the outer periphery 157 of the tabs 154. Other structure and functions of the filter 150 may be similar to the previous embodiment set forth for the filter. For example, similar to the previous embodiment, depicted in FIGS. 4 and 4A, the openings 155 in the filter 150 may be sized and configured to act as a sieve to allow fluid and small particulate to pass but are sized to prevent clumps or larger particulate of a specimen (not shown) to be contained or filtered from solution after being mixed or agitated with the specimen. Further, when the tabs 154 are deflected in the use position or use state in the vile 112 (FIG. 8A), similar to that depicted in FIG. 5A, the spaced distance between the deflected tabs 154 may provide a similar dimension to that of the openings 155. Also, the tabs 154 of the filter in this embodiment may be shorter in length than the previous embodiment as it is contemplated that a shorter length tab may provide more volume for the solution to mix with the specimen, as previously set forth. As shown in FIG. 8A, although the tabs 154 are depicted with a length shorter than a longitudinal length of the loop portion 134 or even less than half the length of the loop portion 134 of the specimen collection member 130, such tabs 154 may extend longer than the longitudinal length of the loop portion 134 of the specimen collection member 130. Importantly, the filter 150 is positioned, at least partially, distal (or on a distal side) of the loop portion 134 of the specimen collection member 130. With this arrangement, after agitating a solution with a specimen in the vile 112 the filter 150 and specimen collection member 130 can be removed from the vile 112 leaving the mixed solution in that same vile 112 originally employed when taking the specimen. Also, as previously set forth, it is contemplated that the filter 150 of this embodiment may be preformed in a use position or, rather, the orientation the filter is positioned when deflected in the vile 112.

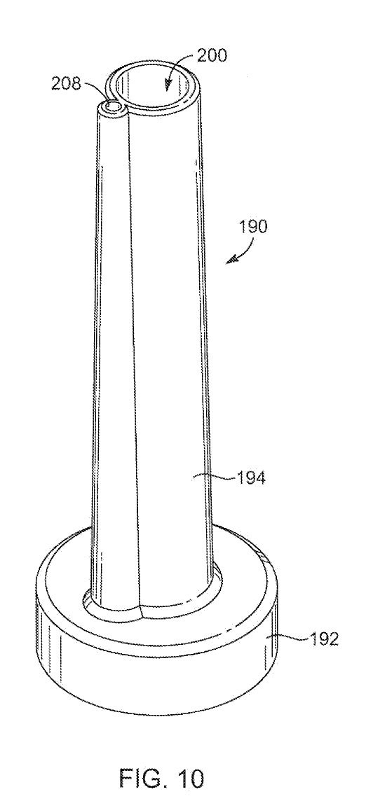

Further, in another embodiment, the lid 190 may include a lid air hole 208, as depicted in FIGS. 8B and 10. As in the previous embodiment, the lid 190 may include a lid head 192 and a lid extension 194, the lid head 192 configured to be captured over the vile 112 and the lid extension 194 defining a hollow portion 200 extending through the lid extension 194 and configured to receive a proximal portion 182 of the handle 132 of the specimen collection member 130. The lid air hole 208 may be defined to extend through a portion of the lid extension 194 and/or the lid head 192. The lid air hole 208 may extend substantially along a longitudinal length of the lid extension 194 and through the lid head 192 that may extend substantially co-axial with the longitudinal axis 160 of the assembled specimen collection assembly 110. In addition, such lid air hole 208 may extend co-axial with the longitudinal axis of the hollow portion 200 defined in and extending through the lid 190. In the assembled arrangement, when a technician inserts a solution through the hollow portion 200 of the lid 190, such solution may pass into the vile 112 along the channels 180 defined in the handle 132 of the specimen collection member 130, as described previously. The lid air hole 208 may be sized and configured to facilitate air passage therethrough as the air in the vile 112 is displaced with solution. As such, the lid air hole 208 may employ means for efficiently inserting the solution into the assembled specimen collection assembly 110. As will be readily recognized by one of ordinary skill in the art, the specimen collection assembly 110 of the embodiments depicted in FIGS. 8A-10 may be employed in the field and/or laboratory in a similar manner and with similar materials as previously set forth for the previous embodiments.

Further, in another embodiment, it is also contemplated that the lid 190 may include a removable plug (not shown) or cover member to cover the upper end of the hollow portion 200 and/or lid air hole 208. Such removable plug may be employed during transport of the assembled specimen collection assembly 110 to assist in maintaining the integrity of the specimen and/or prevent the specimen from being air dried over the duration of being transported from, for example, the field to a laboratory. Once the specimen is in the laboratory and ready for processing, the plug may be removed and discarded for inserting a solution into the vile to be mixed with the specimen.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention includes all modifications, equivalents, and alternatives falling within the spirit and scope of the invention as defined by the following appended claims.

What is claimed is:

1. A method for processing a specimen with a vile having an open end and a closed end, the method comprising:
providing a specimen collection assembly, the assembly including a specimen collection member, a filter and a lid, the specimen collection member having a collection portion and a handle, and the lid configured to cover the open end of the vile;
capturing a specimen with the collection portion of the specimen collection member;
connecting the collection portion to the filter; and
inserting the filter with the connected specimen collection member into the vile so that at least a portion of the filter is positioned distal the collection portion within the vile.

2. The method according to claim 1, further comprising covering the vile with the lid so that a proximal end of the handle extends into a hollow portion defined in the lid.

3. The method according to claim 1, further comprising transporting the specimen collection assembly to another location to analyze the specimen.

4. The method according to claim 2, further comprising inserting a solution through an opening in the lid to at least partially fill the vile with the solution while the lid remains captured over the vile.

5. The method according to claim 2, further comprising inserting a solution through an opening in the lid so that the solution enters the vile by flowing through channels defined in a proximal end of the handle of the specimen collection member.

6. The method according to claim 4, further comprising mixing the solution with the specimen with each of the specimen collection member and the filter captured in the vile and the lid covering the open end of the vile.

7. The method according to claim 6, further comprising removing the specimen collection member and filter from the vile while maintaining interconnection between the specimen collection member and the filter.

8. The method according to claim 7, wherein the removing comprises maintaining the mixed solution in the vile.

9. The method according to claim 1, wherein the inserting comprises deflecting multiple filter portions while inserting the filter through the open end of the vile.

10. The method according to claim 9, wherein the deflecting comprises surrounding at least a distal portion of the collection portion with the filter directly adjacent a distal end of the collection portion and the multiple filter portions deflected and positioned around at least the distal portion of the collection portion.

11. The method according to claim 1, wherein the connecting comprises manually attaching the filter to a distal end of the collection portion of the specimen collection member with a reversible attachment.

12. The method according to claim 1, wherein the connecting comprises inserting an extension portion extending from the filter into a hole defined in a distal end of the collection portion.

13. A method for processing a specimen with a single vile having an open end and a closed end, the method comprising:
providing a specimen captured in a vile with a specimen collection assembly, the specimen collection assembly including a specimen collection member, a filter and a lid, the specimen collection member including a collection portion with the specimen captured therewith the filter removably attached to a distal end of the collection portion and positioned to surround at least a distal portion of the collection portion, and the lid covering an open end of the vile;
inserting a solution through an opening in the lid to at least partially fill the vile;
mixing the solution with the specimen with each of the specimen collection member and the filter captured in the vile and the lid covering the open end of the vile;
removing the specimen collection member and filter from the vile while maintaining interconnection between the specimen collection member and the filter; and
testing the mixed solution from the vile.

14. The method according to claim 13, wherein the providing comprises providing the lid having a hollow portion defined therein for receiving a proximal end of a handle of the specimen collection member.

15. The method according to claim 14, wherein the inserting comprises inserting the solution through the opening and hollow portion defined in the lid so that the solution enters the vile by flowing through channels defined in the proximal end of the handle of the specimen collection member.

16. A specimen collection assembly configured to be used with a container member having an open end and a closed end, comprising:
a specimen collection member having a collection portion and a handle, the collection portion extending from one end of the handle;
a filter sized and configured to be positioned in the container member and configured to be attached to the collection portion in the container member, the filter configured to surround at least a distal portion of the collection portion; and
a lid configured to be positioned over the open end of the container member with the filter and the specimen collection member positioned in the container member.

17. The specimen collection assembly of claim 16, wherein the filter comprises a central portion and a filter portion, the central portion configured to interconnect with the collection portion and the filter portion configured to surround at least the distal portion of the collection portion, the filter portion including multiple openings extending therethrough.

18. The specimen collection assembly of claim 16, wherein the filter comprises a central portion and multiple filter tabs extending from the central portion, the multiple filter tabs configured to be deflectable between a first configuration and a second configuration, the multiple filter tabs configured to be deflectable to the second configuration upon being positioned in the container member.

19. The specimen collection assembly of claim 16, wherein the filter comprises an extension nub extending therefrom configured to attach to a distal end of the collection portion.

20. The specimen collection assembly of claim 19, wherein the collection portion comprises a hole defined therein, the hole configured to receive the extension nub with a snap-type fit.
